(19) United States
(12) Patent Application Publication
(10) Pub. No.: US 2005/0276751 A1
(43) Pub. Date: Dec. 15, 2005

(54) System and method for an automated synthesis of gallium-68 generator-based radiopharmaceutical agents

(76) Inventors: K.S. Clifford Chao, Houston, TX (US); David J. Yang, Sugar Land, TX (US); Firas Mourtada, Pearland, TX (US); Ali Azhdarinia, Friendswood, TX (US)

Correspondence Address: FULBRIGHT & JAWORSKI L.L.P., 600 CONGRESS AVE., SUITE 2400, AUSTIN, TX, 78701, US

(21) Appl. No.: 11/038,631

(22) Filed: Jan. 19, 2005

Related U.S. Application Data
(60) Provisional application No. 60/538,191, filed on Jan. 20, 2004.

(51) Int. Cl. A61K 51/00; C07F 5/00

(52) U.S. Cl. 424/1.11; 534/11

(57) ABSTRACT

Systems and methods are described for efficiently producing a radiopharmaceutical agent. The system includes a generator that provides an admixture comprising hydrochloric acid and gallium-68 (Ga-68). A heater initiates a removal process by evaporating the hydrochloric acid from the admixture resulting in a substantially purified Ga-68. A plurality of valves provides a buffer and a prodrug that gets mixed with the Ga-68 to produce Ga-68 radiopharmaceutical agent. Additionally, the valves may provide water or a transchelator to the Ga-68 radiopharmaceutical agent to optimize the yield. A method includes providing an admixture comprising hydrochloric acid and Ga-68. The method also includes removing the hydrochloric acid by heating the admixture and subsequently evaporating the hydrochloric acid, resulting in a substantially purified Ga-68. The method further includes adding a buffer and a prodrug to the substantially purified Ga-68 to produce the Ga-68 radiopharmaceutical agent.

This patent application claims priority to, and incorporates by reference in its entirety, U.S. provisional patent application Ser. No. 60/538,191 filed on Jan. 20, 2004.

### BACKGROUND OF THE INVENTION

1. Field of the Invention

The invention relates generally to the field of radiotracer synthesizers. More particularly, the invention relates to a system and a method for producing gallium-68 radiopharmaceutical agents.

2. Discussion of the Related Art

Positron emission tomography (PET) is an in vivo imaging method which uses gamma radiotracers to track the biochemical, molecular, and/or pathophysiological processes in humans and animals. In PET systems, positron-emitting isotopes serve as beacons for identifying the exact location of diseases and pathological processes under study without surgical exploration of the human body. With these non-invasive imaging methods, the diagnosis of diseases may be more comfortable for patients, as opposed to the more traditional and invasive approaches, such as exploratory surgeries.

Currently, some of the available radiotracers are produced from a cyclotron (F-18) process. A cyclotron system accelerates charged particles to high speeds and causes these charged particles to collide with a target to produce a nuclear reaction and subsequently create a radioisotope. However, the cyclotron-based tracers are constrained by the availability of local cyclotron and the cost of production.

Another method for producing radiotracers is through use of a generator process. The generator process uses a parent-daughter (P/D) nucleic pair where the parent (P) isotope decays to a short-lived daughter (D) isotope used for imaging. However, the current generator-based radiotracers are limited by the half-life of radioisotopes and the limited choices of imaging agents. For example, the copper-62 generator produces a Cu-62 based radioisotope with a half life of less than 10 minutes. As known in the art, radiosynthesis of radiotracers must be rapid because the usable amount of the radioisotope will decay with lengthy chemical synthesis and can cause a higher risk of radiation exposure during the production process.

The referenced shortcomings are not intended to be exhaustive, but rather are among many that tend to impair the effectiveness of previously known techniques concerning the production of radiotracers; however, those mentioned here are sufficient to demonstrate that the methodologies appearing in the art have not been satisfactory and that a significant need exists for the techniques described and claimed in this disclosure.

### SUMMARY OF THE INVENTION

The present invention provides a system and method for producing an isotope with a prolonged half-life and producing both water and lipid soluble PET tracers in an efficient manner. Further, the invention produces an isotope that permits a more comprehensive radiochemistry for a variety of PET imaging agents.

In one respect, a method for producing a gallium-68 (Ga-68) radiopharmaceutical agent is provided. The method may include providing an admixture of Ga-68 and hydrochloric acid. The hydrochloric acid may subsequently be removed by an evaporation process in which the admixture may be heated to approximately 95-105° Celsius for approximately 10 to 15 minutes, yielding substantially purified Ga-68. In one embodiment of the invention, the admixture may be heated to approximately 100° Celsius.

The substantially purified Ga-68 may be mixed with a buffer and a prodrug to produce a Ga-68 radiopharmaceutical agent. In one embodiment of the invention, the buffer may be a sodium-acetate buffer. To facilitate the mixing of buffer and prodrug with the substantially purified Ga-68, nitrogen may be provided. In another embodiment, a carrier, for example, GaCl₃ may also be provided to the substantially purified Ga-68 to produce the radiopharmaceutical agent.

In another respect, a system is provided. The system includes a generator, such as a Ga-68 generator which provides an admixture including Ga-68 and hydrochloric acid. The system may also include a heater for heating the admixture, which may be stored in a mixing chamber. The heater may heat the admixture to approximately 95-105° Celsius for approximately 10 to 15 minutes to induce the evaporation of the hydrochloric acid, leaving substantially purified Ga-68. In one embodiment, the heater heats the admixture to approximately 100° Celsius

The system may also include a valve assembly that may at least provide a buffer, for example, a sodium acetate buffer, a carrier, for example, GaCl₃, and a prodrug to mix with the purified Ga-68 to produce a Ga-68 radiopharmaceutical agent. Nitrogen may be provided via a valve within the valve assembly to aid the mixing of the buffer, prodrug, and the purified Ga-68. In one embodiment, the mixing process may be captured by a camera coupled to the mixing chamber, where the camera may provide images from the mixing to a processor coupled to the camera to ensure quality control of the synthesis process.

The valve assembly and the heater may be operably controlled by a control system. The control system may receive instructions from a computer programmable software that may facilitate the operations of the system.

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The drawings accompanying and forming part of this specification are included to depict certain aspects of the invention. A clearer conception of the invention, and of the components and operation of systems provided with the invention, will become more readily apparent by referring to the exemplary, and therefore nonlimiting, embodiments illustrated in the drawings, wherein like reference numerals (if they occur in more than one view) designate the same or similar elements. The invention may be better understood by reference to one or more of these drawings in combination with the description presented herein. It should be noted that the features illustrated in the drawings are not necessarily drawn to scale.

FIG. 1A is a block diagram of a system for producing radiopharmaceutical agents in accordance with an embodiment of the present invention.

FIG. 1B is a block diagram of a control system in accordance with an embodiment of the present invention.

FIG. 2 is a detailed block diagram of a system for producing radiopharmaceutical agents in accordance with an embodiment of the present invention.

FIG. 3A is a software simulation of the system of FIG. 2 for controlling the operations of the system components in accordance with an embodiment of the invention.

FIG. 3B is a software program that controls the inputs to the system in accordance with an embodiment of the invention.

FIG. 4 is a software program for controlling the operations of a system in accordance with an embodiment of the invention.

FIG. 5 is an example of a computer programmable software program in accordance with an embodiment of the invention.

FIG. 6 is a flowchart showing steps of a method in accordance with one embodiment of the invention.

### DETAILED DESCRIPTION

The invention and the various features and advantageous details thereof are explained more fully with reference to the nonlimiting embodiments that are illustrated in the accompanying drawings and detailed in the following description. It should be understood that the detailed description and the specific examples, while indicating specific embodiments of the invention, are given by way of illustration only and not by way of limitation. Various substitutions, modifications, additions and/or rearrangements within the spirit and/or scope of the underlying inventive concept will become apparent to those of ordinary skill in the art from this disclosure.

The present invention includes a system and a method for the synthesis of gallium-68 (Ga-68) radiopharmaceutical agents used in imaging methods, such as Positron Emission Tomography (PET) imaging. The use of Ga-68 may permit a more comprehensive radiochemistry due to the 68-minute half-life of Ga-68. Further, the system and method of the present invention may be capable of producing water and lipid PET tracers with at least a 95% yield, if not 100% yield, at an efficient rate, e.g. less than 20 minutes.

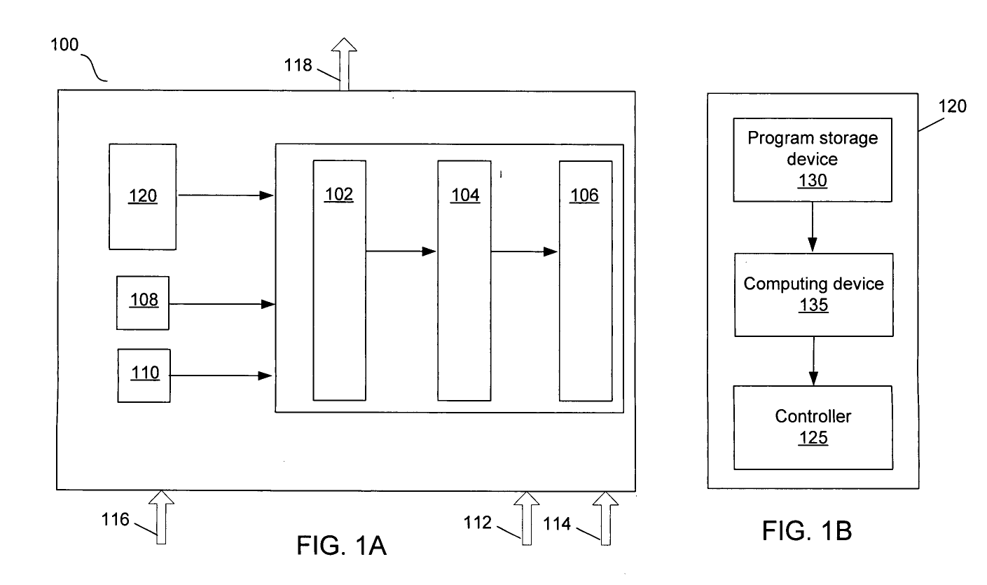

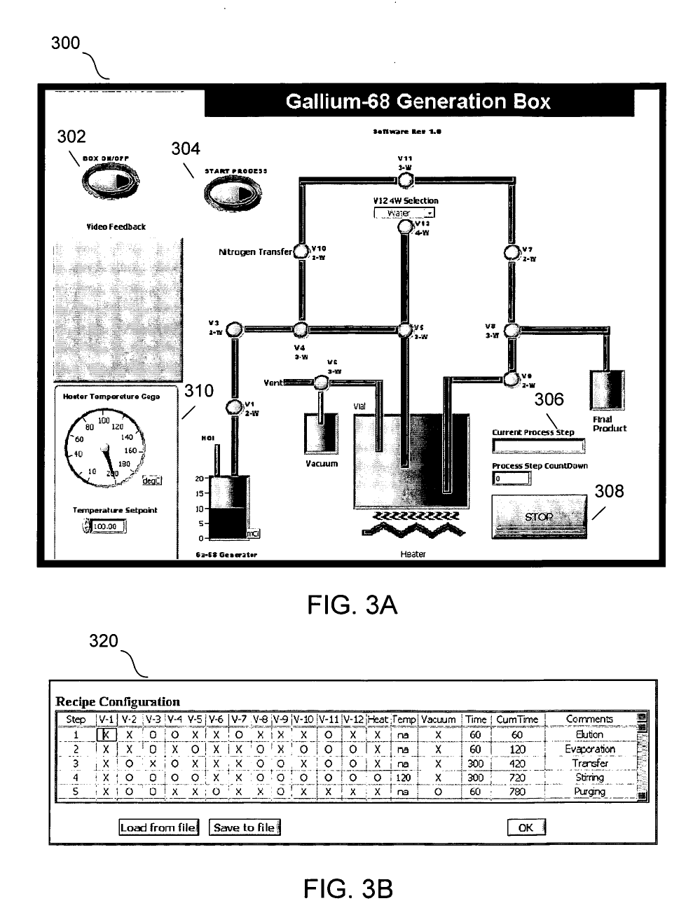

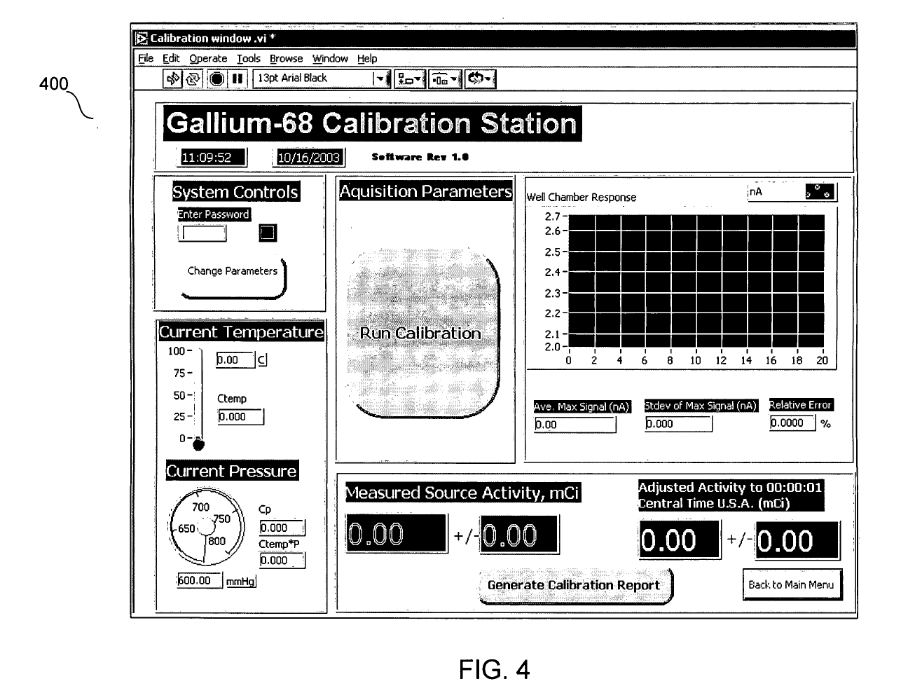

Referring to FIG. 1A, a block diagram of a system 100 for producing Ga-68 radiopharmaceutical agents is presented. In one embodiment, system 100 may be self-contained unit mounted onto a hooded tabletop. Alternatively, system 100 may be integrated into a multi-purpose automated radiotracer synthesizer, as disclosed in U.S. patent application Ser. No. 10/982,530, filed Nov. 5, 2004, and entitled “Multi-Purpose Automated Radiotracer Synthesizer”, expressly incorporated here by reference. System 100 may include at least three compartments (102, 104, and 106). Each compartment may house a plurality of components, including, but not limited to, a valve assembly, vents, chambers, etc. System 100 may also include generator 104. In one embodiment, the generator may be a Ga-68 generator, which may have the dimensions of about 10.5 inches tall and a diameter of 5.5 inches. The generator may be encased in lead and may include a connection that outputs an admixture of Ga-68 and hydrochloric acid to the system 100. Coupled to the generator may be a heater 110. The heater 110 may comprise a control system 120 used for feedback purpose and may initiate the purification of the Ga-68 by removing, the hydrochloric acid from the admixture. The features of the control system 120 will be further discussed below with reference to FIG. 3A, FIG. 3B, and FIG. 4.

System 100 may also include a plurality of inputs 112, 114, and 116. Each input may provide the system 100 with chemical compounds, drugs, and/or other components needed to produce a radiopharmaceutical agent. For example, input 112 may provide nitrogen (N₂) to aid in the transfer and/or mixing of the chemical compounds, drugs, and/or other products through the system 100. Input 114 may provide a vacuum pump which may be used to create a vacuum in one of the chambers, such as a mixing chamber of the system. Input 116 may provide a prodrug to the system to produce the radiopharmaceutical agent. System 100 may include at least one output, such as a Ga-68 radiopharmaceutical agent. The functionality of each of the components of system 100 will further be discussed below.

Coupled to system 100 may be a control system 120, which may include a computing device 135, a program storage media 130, and a controller 125, as shown in FIG. 1B. The computing device 135 may be, for example, a personal computer or a laptop computer. The computing device 135 may also be a programmable circuit, such as, for example, a microprocessor or digital signal processor-based circuit, that operates in accordance with instructions stored in the program storage media 130. The program storage media 130 may be any type of readable memory including, for example, a magnetic or optical media such as a card, tape or disk, or a semiconductor memory such as a PROM or FLASH memory. The controller 125 may be, for example, a programmable logic controller. The computing device 135 may execute a program of instructions stored in a program storage media 130 and sends commands to the controller 125. As such, system 100 may be configurable, reconfigurable, and controllable via a software graphical user interface (GUI) depending on the configuration.

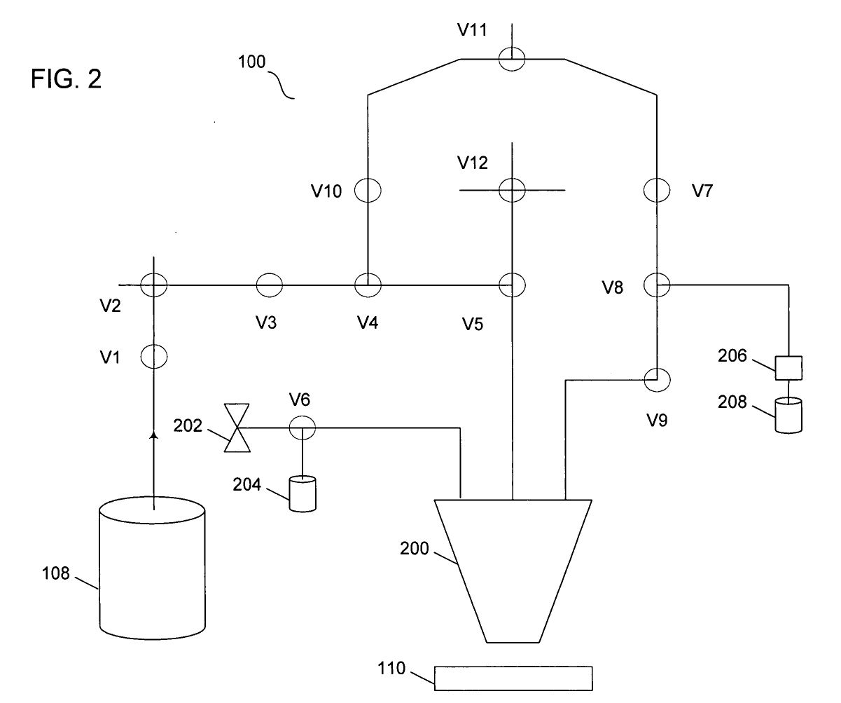

FIG. 2 is a detailed illustration of system 100 for producing a radiopharmaceutical agent. In particular, system 100 may include a valve assembly comprising valves V1 through V12 which may control the flow of the inputs 112, 114, and 116 and others throughout the system. In one embodiment, the valves may be a series of two, three, and/or four-way valves. The system may also include a mixing chamber 200 in which the mixing chamber 200 may be used to mix a radioisotope with other components to produce a radiopharmaceutical agent. Attached to the mixing chamber 200 may be a camera (not shown) that may provide visual feedback of the mixing process within a system to a display device of a computer system.

The synthesis process begins with the production of Ga-68 from the generator 108. During the synthesis, an acid, such as hydrochloric acid, may be added to produce the Ga-68. Due to the harmful effects of the hydrochloric acid to a patient, there is a need to remove the hydrochloric acid before producing the radiopharmaceutical agent.

The valves in the valve assembly may be controlled by controller 120 to be “ON” position providing an input or transferring a product, or an “OFF” position where no transfer through the system occurs at that valve. In one embodiment, the generator 108 may provide an admixture which may comprise Ga-68 and hydrochloric acid. The admixture may be routed to the mixing chamber 200 (V2, V2, V4, and V5 “ON”). In addition, a vent 202 may be utilized (V6 “ON”). To ensure a sterile condition and no aqueous back flow to the generator 108, after the transfer of the admixture to the mixing chamber 200, V4, V3, V2 and V1 may be switched to an “OFF” position.

Next, acetonitrile may be added to the admixture in the mixing chamber 200 for an azeotropic evaporation of the hydrochloric acid. The heater 110, which may be coupled to the mixing chamber 200, may be turned on to approximately 95-105° Celsius, under vacuum (V6 “ON”) and nitrogen (V7, V8, V9, and V11 “ON”) to begin the removal of the hydrochloric acid from the admixture. The heater remains on for approximately 10-15 minutes to ensure the complete evaporation of the hydrochloric acid leaving substantially purified Ga-68 in the mixing chamber 200. After the evaporation, the mixing chamber 200 may be cooled down by adding nitrogen for approximately 30 seconds (heater 110 “OFF”; V7, V8, V9 and V11 “ON”).

To form the Ga-68 radiopharmaceutical agent, a buffer, such as a sodium acetate buffer, a carrier, and a prodrug may be added to the mixing chamber 200 (V9 “OFF”, VS and V12 “ON”). In one embodiment, the carrier may be cold or un-labeled gallium-chloride (GaCl₃) at a specific molar concentration, such as 4 mM. The substantially purified Ga-68, buffer, carrier, and prodrug may be stirred by providing nitrogen to the mixing chamber 200 (V5 “OFF” and V9 “ON”). The Ga-68 radiopharmaceutical agent may be subsequently transferred and stored in a sterile container, such as the collection chamber 208 (V6 and V7 “OFF”; V5, V4, V8, and V9 “ON”; and V10 and VII “ON”). The collection chamber 208 may be made of lead and may include a door (not shown) to provide access to the product stored within the collection chamber 208.

Once the product is transferred and stored in the collection chamber 208, water, a carrier, such as GaCl₃, or transchelator may be added (V4 and V9 “OFF”; V5 and V6 to the vent “ON”) to the mixing chamber and may be subsequently transferred to the collection chamber 208 to optimize the yield of the radiopharmaceutical agent (V6 “OFF”; V5, V4, and V9 “ON”).

The operation of system 100 may be controlled by control system 120 (FIG. 1). In one embodiment, the control system 120 may include a digital output module used to control the operations of the valve assembly of system 100. For example, each valve in the valve assembly may be solenoid actuated with an applied voltage of approximately 24 Volts from the control system. In the disclosed embodiment, the control system 120 may include software modules written to interact with a FieldPoint, a control system available from National Instruments. However, it will be understood that other control systems would also be acceptable.

Referring to FIG. 3A, system 100 is emulated in software, in which the software may provide instructions to a controller of the control system 120. A software window 300, such as a graphical user interface (GUI), may be displayed on a display device, such as a monitor, printer, etc., of a processing unit and may allow monitoring and control to a user during the synthesis phase. In the disclosed embodiment, the software emulation is based on a GUI written in LabVIEW, available from National Instruments. However, it will be understood that other forms of software would also be acceptable. Software window 300 may display a plurality of controls. Control 302 may power the system 100. Control 304 may begin the process of producing the radiopharmaceutical agent. Software window 300 may also display a process window 306, which may display the process in which the system is currently operating, e.g., evaporation, mixing, transferring, etc. Further, software 300 may also display control 308, which may halt the process of the system at any time. In one embodiment of the invention, a calibration report of system 100 may be generated via a calibration station, as illustrated in FIG. 4. The calibration station software window 400 may include a plurality of displays showing the different parameters of system 100. The parameters may include, but is not limited to, current temperature and pressure of the system.

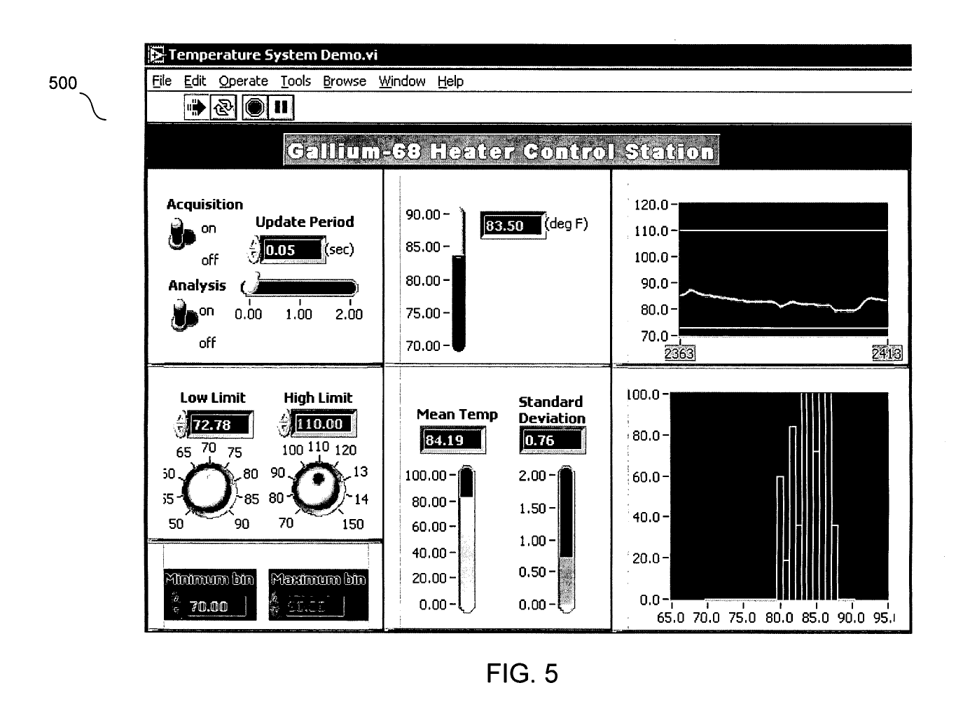

Software window 300 may also display control 310 which is temperature gauge that monitors the heat emitted from the heater as well as allow the setting of a heater to a desired temperature, e.g., 100° Celsius. In addition, the software program may also provide a more detailed analysis of the heater as illustrated in FIG. 5. Software window 500 may include a plurality of displays including analysis generated from feedback signals provided to the control system of system 100.

In one embodiment of the invention, the software may provide a display window for configuring system 100. Referring to FIG. 3B, display window 320 may provide a user editing control to the “recipe” that produces the radiopharmaceutical agent. In one embodiment, the display window may allow for modifying existing recipes or create new recipes. By determining what valves need to be turned for a certain time period, which may allow the flow of inputs such as prodrugs, buffers, nitrogen, etc. through the system, the synthesis of the radiopharmaceutical agent may be automated. After the “recipe” has configured, the software program may provide the configurations to the control system 120 and the process may be initiated.

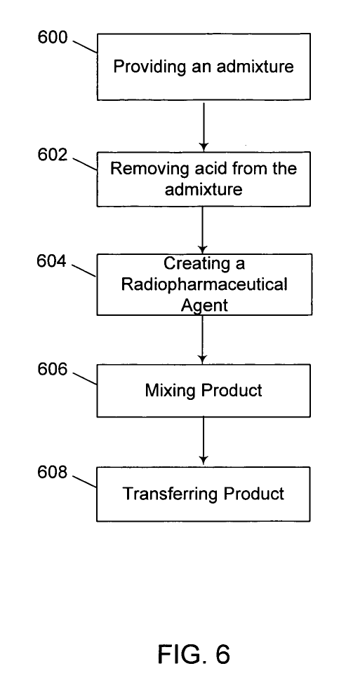

In another embodiment of the invention, a method may be provided for producing a radiopharmaceutical agent. Referring to FIG. 6, an admixture may be provided by a generator any may comprise Ga-68 and an acid, such as hydrochloric acid (step 600). Next, the acid may be removed from the admixture (step 602). In one embodiment, acentonitrile may be included to the admixture to facilitate an azeotropic evaporation of an acid. In addition, the admixture may be heated which may initiate the evaporation of the acid leaving substantially purified Ga-68. The substantially purified Ga-68 may be combined with other products to form a radiopharmaceutical agent (step 604). The products may comprise a buffer, e.g., sodium acetate buffer, and/or a prodrug. The combination of the producst and the substantially purified Ga-68 may be mixed aided by nitrogen (step 606). The radiopharmaceutical agent may then be transferred and stored (step 608). Additionally, water, a carrier, and/or a transchelator may be added to the radiophamaceutical agent to optimize the yield.

Although the present invention and its advantages have been described in detail, it should be understood that various changes, substitutions and alterations can be made to the embodiments herein without departing from the spirit and scope of the invention as defined by the appended claims. Moreover, the scope of the present application is not intended to be limited to the particular embodiments of the process, machine, manufacture, and composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A method for producing a gallium-68 radiopharmaceutical agent, comprising:
providing an admixture of gallium-68 and an acid;
removing the acid from the admixture to produce substantially purified gallium-68; and
mixing the substantially purified gallium-68 with a buffer and a prodrug to produce a gallium-68 radiopharmaceutical agent.

2. The method of claim 1, the acid comprising hydrochloric acid.

3. The method of claim 1, the step of removing comprising evaporating the hydrochloric acid.

4. The method of claim 3, the step of removing further comprising adding acetonitrile to the admixture for an azeotropic evaporation of the hydrochloric acid.

5. The method of claim 3, the step of evaporating further comprising heating the admixture to approximately 95-105° Celsius.

6. The method of claim 3, the step of heating further comprising heating the admixture for approximately 10 to 15 minutes.

7. The method of claim 1, the buffer comprising a sodium-acetate buffer.

8. The method of claim 1, the step of mixing further comprising adding a carrier to produce the Ga-68 radiopharmaceutical agent.

9. The method of claim 8, the carrier comprising gallium chloride.

10. The method of claim 1, the step of mixing further comprising adding nitrogen to for mixing of the substantially purified gallium-68 with the buffer and the prodrug .

11. The method of claim 1, further comprising adding water to the gallium-68 radiopharmaceutical agent to optimize the yield of the gallium-68 radiopharmaceutical agent.

12. The method of claim 1, further comprising adding a transchelator to the gallium-68 radiopharmaceutical agent to optimize the yield of the gallium-68 radiopharmaceutical agent.

13. The method of claim 1, further comprising adding a carrier to the gallium-68 radiopharmaceutical agent to optimize the yield of the gallium-68 radiopharmaceutical agent.

14. The method of claim 13, the carrier comprising gallium chloride.

15. A computer program, comprising computer or machine-readable program elements translatable for implementing the method of claim 1.

16. A system, comprising:
a generator for providing an admixture comprising an acid and gallium-68;
a mixing chamber coupled to the generator, the mixing chamber storing the admixture;
a heater coupled to the mixing chamber, the heater initiating an evaporation process of the acid to produce substantially purified gallium-68; and
a plurality of valves coupled to the mixing chamber, the plurality of valves providing a buffer and a prodrug to the substantially purified gallium-68 to produce gallium-68 radiopharmaceutical agent.

17. The system of claim 16, the acid comprises hydrochloric acid.

18. The system of claim 16, the generator comprising a gallium-68 generator.

19. The system of claim 16, the heater heating the admixture to approximately 95-105° Celsius.

20. The system of claim 19, the heater heating the admixture to approximately 100° Celsius.

21. The system of claim 19, the heater heating the admixture for approximately 10 to 15 minutes.

22. The system of claim 16, further comprising a valve for providing a carrier to produce the Ga-68 radiopharmaceutical agent.

23. The system of claim 22, the carrier comprising gallium chloride.

24. The system of claim 22, further comprising a valve for providing nitrogen to the mixing chamber for mixing the buffer, the prodrug, the carrier, and the substantially purified gallium-68.

25. The system of claim 16, further comprising a control system for operating the valves.

26. The system of claim 25, the control system controlling the heater to operate at a desired temperature.

27. The system of claim 25, the control system comprising a computer programmable software for providing instructions to the control system.

28. The system of claim 16, further comprising a collection chamber for storing the gallium-68 radiopharmaceutical agent.

29. A program storage device readable by a machine, tangibly embodying a program of instructions executable by the machine to perform the method steps for producing a radiopharmaceutical agent, the method steps comprising:
providing an admixture of gallium-68 and hydrochloric acid;
removing the hydrochloric acid from the admixture to produce purified gallium-68; and
mixing the purified gallium-68 with a buffer, a prodrug, and a carrier to produce a gallium-68 radiopharmaceutical agent.

30. The program storage device of claim 29, the buffer comprising a sodium-acetate buffer.

31. The program storage device of claim 29, the carrier comprising gallium chloride.
